(19) United States
(12) Patent Application Publication
(10) Pub. No.: US 2006/0046245 A1
(43) Pub. Date: Mar. 2, 2006

(54) Porous device

(75) Inventor: Ryszard Kobylecki, Cambridgeshire (GB)

Correspondence Address: NIXON & VANDERHYE, PC, 901 NORTH GLEBE ROAD, 11TH FLOOR, ARLINGTON, VA, 22203, US

(73) Assignee: Millennium Pharmaceuticals Limited, Cambridge (GB)

(21) Appl. No.: 11/240,386

(22) Filed: Oct. 3, 2005

Related U.S. Application Data
(63) Continuation of application No. 09/807,504, filed on Dec. 17, 2001.

(30) Foreign Application Priority Data
Oct. 14, 1998 (GB) 9822436.3

(51) Int. Cl. C40B 60/08; C12Q 1/00; C12P 21/06

(52) U.S. Cl. 435/4; 435/289.1; 435/69.1

(57) ABSTRACT

A porous device for use in a method of synthesis comprising a body having an internal region which is porous wherein an active material, for example a solid support resin, is entrapped within the internal region.

This invention relates to a porous device. Preferred embodiments relate to the use of a porous device or porous devices for example in a method of synthesis (especially a “mix and split”, parallel array or combinatorial method); a porous device per se; and a method of manufacturing a porous device.

The use of solid supports in the synthesis of compounds for example peptides or any other types of chemical compounds is well-known. One particularly important contribution to this field was made in 1963 by Merrifield who disclosed the preparation of spherical styrene-divinyl benzene beads for use in synthesis. A wide range of other functionalized beads have been proposed since, as have methods and devices for handling the beads and/or using them in the automated synthesis of libraries of compounds For example, beads have been provided in the form of “tea bags”. A more sophisticated format utilizes porous re-usable tubes into which samples of resin are weighed, for example as described in WO96/36436 (see for example FIG. 14). Disadvantageously, the tubes are expensive and furthermore, need to be charged with carefully weighed out samples of resin which can be time-consuming. In addition, the tubes themselves generally have an internal volume which is significantly greater than the volume of resin incorporated in the tubes, thereby to allow for the swelling of the resin (up to 3 or 4 times its original volume) in solvents with which the tubes may be contacted in synthesis procedures.

It is an object of the present invention to address the above described problems.

According to a first aspect of the present invention, there is provided a method of synthesis using a porous device comprising a body having an internal region which is porous, wherein an active material is entrapped within the internal region.

Preferably, in the method, a covalent bond is formed between the active material and a reagent (or a fragment thereof) used in said synthesis.

Preferably, the method includes the step of contacting is said porous device with a first reagent under conditions which cause said first reagent to react with said active material, so that a bond, preferably a covalent bond, is formed between the active material and said first reagent (or a fragment thereof). Preferably, the method further includes contacting the porous device with a second reagent under conditions which cause said second reagent to react with the first reagent (or a fragment thereof) bonded to the active material. As a result, said second reagent (or a fragment thereof) may be bonded, preferably covalently bonded, to said first reagent (or a fragment thereof). Thus, said method preferably involves contacting, preferably sequentially, said porous device with reagents (e.g a said first reagent, a said second reagent etc) in order to prepare a compound which is covalently bonded to the active material of the porous device. Thus, said method preferably involves contacting said porous device with at least two, preferably at least three, more preferably at least four, reagents wherein each reagent interacts with one of either the active material and/or a moiety bonded to the active material, in order to facilitate the making or breaking of covalent bonds.

Said active material preferably includes a functional group which is not inert but which is reactive. For example, said functional group is preferably able to react in nucleophilic or electrophilic reactions. Said active material is preferably arranged to act as a support for a compound prepared in solid phase synthesis. Said active material is preferably not a catalyst (and/or does not function as a catalyst in said method) for catalysing solution or gas phase reactions.

Said active material preferably includes a functional group selected from a chloromethyl, hydroxymethyl or aminomethyl group or a derivative thereof.

Said active material may include a linker or may be covalently bonded to a linker in said synthesis, for example in a first step thereof, wherein said linker comprises a moiety to which a compound being prepared in said synthesis is bonded during its synthesis and wherein after preparation of said compound, it may be cleaved from the active material by breaking a bond between the linker and said compound. Methods of providing linker moieties are well-known. Examples are provided in Tetrahedron Vol. 51, No. 30, pages 8135 to 8173 (1995) (the contents of which are incorporated herein by reference) and may include Wang, Rink and Trityl Linkers.

Thus, said method preferably include the step of cleaving a compound prepared from the active material. The compound cleaved may then be isolated. In the method, the active material may be contacted with at least two, suitably at least three, preferably at least four, more preferably at least five, different compounds (which term includes any solvents or reactants) prior to said cleavage step.

Said internal region is preferably unitary. Said internal region is preferably monolithic. Said internal region preferably does not include a plurality of distinct layers of material. Suitably, material(s) which make(s) up said internal region is/are fixed in position in said internal region. The arrangement and/or position of the active material (e.g particles thereof) is preferably predetermined. Said material(s) is/are preferably fixed in position by a means within said internal region. Said internal region of said body preferably has a predetermined shape. Said predetermined shape may be varied, for example due to said internal region being flexible. However, the shape of said internal region is preferably substantially fixed. Said internal region is preferably not flowable, for example at 25° C. Said internal region suitably comprises a random network of pores which preferably has a substantially fixed configuration and which suitably extends throughout substantially the entirety of said internal region. Said active material is preferably distributed throughout substantially the entirety of said internal region. Said network of pores is preferably not defined by a fabric and/or a filamentous and/or a fibrous material. Said internal region preferably does not include a fabric and/or a filamentous and/or a fibrous material. Said internal region is preferably arranged for passage of fluid from one side to an opposite side thereof. Substantially the entirety of said internal region is porous. Preferably, the porosity of the internal region is substantially constant across its extent. Preferably, the void volume of the internal region is substantially constant across its extent. Said void volume of said internal region may be at least 20%, suitably at least 30%, preferably at least 40%, more preferably at least 45%, especially at least 48%. Said void volume may be less than 80%, suitably less than 70%, preferably less than 60%, more preferably less than 55%, especially 52% or less.

Said internal region may extend substantially uninterruptedly, in a first direction from one outer wall of the porous device to an opposite outer wall thereof. Said internal region may extend substantially uninterruptedly in a second direction (perpendicular to said first direction) from one outer wall of the porous device to an opposite outer wall thereof. Said internal region may extend substantially uninterruptedly in a third direction (perpendicular to said first and second directions) from one outer wall of the porous device to an opposite outer wall thereof. Alternatively, an opening, for example a hollow region, may be defined within the body of the porous device which hollow region may extend from one outer wall of the porous device to an opposite outer wall thereof. For example, where the porous device is cylindrical, it may have an open-ended cylindrical hollow region extending in the direction of the axis of the cylinder. The provision of an opening as described may increase the surface area of the internal region which can be contacted with fluid in said method of synthesis. In this event, the shape of the internal region may be selected to maximise the available surface area—for example it may have a star or other convoluted shape. In another situation, the opening may be arranged to accommodate an identification means as hereinafter described.

Preferably, said internal region includes at least one sinterable material. Said sinterable material may be a thermoplastic. Said internal region preferably includes at least one sintered material. Said sinterable material preferably defines the porous structure of said internal region.

Said internal region may have a dimension in a first direction of at least 1 mm, suitably at least 2 mm, preferably at least 3 mm, more preferably at least 4 mm, especially at least 5 mm. Said internal region may have a dimension in a second direction, perpendicular to said first direction, of at least 1 mm, suitably at least 2 mm, preferably at least 3 mm, more preferably at least 4 mm, especially at least 5 mm. Said internal region may have a dimension in a third direction, perpendicular to said first and second directions, of at least 1 mm, suitably at least 2 mm, preferably at least 3 mm, more preferably at least 4 mm, especially at least 5 mm. Preferably, at least one (more preferably two, especially three) of said first, second or third directions is/are coincident with a respective axis of symmetry of the internal region.

Said porous device preferably has a predetermined shape. Said porous device may be flexible. However, said shape of said porous device is preferably fixed. Said porous device suitably comprises a random network of pores which preferably has a substantially fixed configuration. Said porous device is preferably not of a layered or sandwich construction. Thus, it preferably does not include a plurality of distinct layers of material. It preferably comprises a single unitary material (which may itself be made up of a mixture of one or more components)—that is, said porous device is preferably substantially monolithic. Said porous device is preferably arranged for passage of fluid in a first direction from one side to an opposite side thereof. Thus, pores of said internal region suitably open at surfaces of the device. Preferably, said porous device is arranged for passage of fluid in a second direction, perpendicular to said first direction, from one side to an opposite side thereof. Preferably, said porous device is arranged for passage of fluid in a third direction, perpendicular to both said first and second directions, form one side to an opposite side thereof. Preferably, said porous device is freely porous in three mutually perpendicular directions. Suitably, at least 50%, preferably at least 75%, more preferably at least 90%, especially at least 95% of the surface of the device is porous. Most preferred is the case wherein substantially the entirety of the outer surface of said device is porous. Preferably, the porosity of the porous device is substantially constant across its extent. Preferably, the void volume of the porous device is substantially constant across its extent. Said void volume of said porous device may be at least 20%, suitably at least 30%, preferably at least 40%, more preferably at least 45%, especially at least 48%. Said void volume may be less that 80%, suitably less than 70%, preferably less than 60%, more preferably less than 55%, especially 52% or less.

Preferably, the porosity at a surface of the device is substantially the same as the porosity of the internal region adjacent said surface. Preferably, some active material is at or adjacent the surface of the porous device and is, suitably, in a fixed position relative to the surface. Said porous device preferably does not include any fabric and/or filamentous and/or fibrous material.

Said porous device may be provided in any desired shape and, more particular, in any shape that has been proposed to be used as a solid support in solid support reactions. For example, said device may be in the form of a cylinder, rod, sheet, capsule, tablet, plug, disc, streamer or tape. Preferred shapes have a smallest dimension of at least 1 mm, suitably at least 2 mm, preferably at least 3 mm, more preferably at least 4 mm, especially at least 5 mm. Preferred shapes of said device include cylinders, rods, capsules, tablets or plugs. Any porous device may include an appendage, for example a hook, opening (or the like) to enable the device to be picked up and put down, preferably robotically. An especially preferred shape of a porous device may be as described in WO96/36436 (e.g. see FIG. 14) and the shapes described are incorporated herein by reference. Advantageously, the use of the aforementioned shapes may allow existing apparatus to be used to manipulate the porous device.

Said porous device is preferably substantially self-supporting. Said porous device is preferably substantially rigid.

In one embodiment, the porous device may be provided in a sheet form which is used to support a multiplicity of spot syntheses. Preferably, however, said device is not a sheet.

Said porous device may have a dimension in a first direction of at least 1 mm, suitably at least 2 mm, preferably at least 3 mm, more preferably at least 4 mm, especially at least 5 mm. Said dimension in said first direction may be less than 100 mm, suitably less than 80 mm, preferably less than 50 mm, more preferably less than 30 mm, especially less than 10 mm. Said porous device may have a dimension in a second direction, perpendicular to said first direction, of at least 1 mm, suitably at least 2 mm, preferably at least 3 mm, more preferably at least 4 mm, especially at least 5 mm. Said dimension in said second direction may be less than 100 mm, suitably less than 80 mm, preferably less than 50 mm, more preferably less than 30 mm, especially less than 10 mm. Said porous device may have a dimension in a third direction, perpendicular to said first and second directions, of at least 1 mm, suitably at least 2 mm, preferably at least 3 mm, more preferably at least 4 mm, especially at least 5 mm. Said dimension in said third direction may be less than 100 mm, suitably less than 80 mm, preferably less than 50 mm, more preferably less than 30 mm, especially less than 10 mm. Preferably, at least one (more preferably two, especially three) of said first, second or third directions is/are coincident with a respective axis of symmetry of the porous device.

Said internal region preferably makes up at least 30%, suitably at least 50%, preferably at least 70%, more preferably at least 90%, especially at least 95%, of the volume of said porous device. The volume of the internal region is preferably substantially the same as the volume of the porous device.

Said porous device (suitably said internal region of said body) may have a volume of at least 25 mm², preferably at least 50 mm², preferably at least 100 mm², more preferably at least 150 mm², especially at least 200 mm². Said volume may be less than 1000 mm², suitably less than 5000 mm², preferably less than 2500 mm², more preferably less than 1000 mm² especially less than 500 mm².

The porosity of the device to methanol at ambient temperature and pressure may be at least 0.2 ml/minute, suitably at least 0.4 ml/min, preferably at least 0.6 ml/min, especially at least 0.8 ml/min. Said porosity may be less than 1.5 ml/min, preferably less than 1 ml/min.

Said porous device may include at least 10 μmol, suitably at least 25 μmol, preferably at least 40 μmol, more preferably at least 55 μmol, especially at least 70 μmol of functionality available for participation in the synthesis.

Said internal region of said porous device, for example pores thereof, may be defined by active material so that, suitably, said internal region may consist essentially of active material. The active material may comprise a single material or a plurality of different active materials may be included. Such a porous device is referred to hereinafter as a “first type” of porous device. Another type of porous device, hereinafter referred to as a “second type” of porous device, may comprise an inert material (in the sense that it is not covalently bonded to a compound synthesized in the method) and an active material. The inert material may be arranged to entrap the active material within the internal region of the device. Thus, said inert material may define a porous support means and said active material may be arranged within pores of said porous support means. Preferably, said active material is not covalently bonded to said porous support means. Preferably, the majority (e.g greater than 50%, suitably greater than 60%, preferably greater than 70%, more preferably greater than 80%, especially greater than 90%) of said active material is exposed (i.e. not covered by inert material) so that the majority of said active material is available for bond formation in said synthesis. Preferably, said porous support means is not a fabric and/or filamentous and/or fibrous material. Preferably, the active material is in the form of a multiplicity of individual particles, wherein said particles are separated from one another by said inert material and, suitably, are held in substantially fixed positions relative to one another and/or relative to said inert material. Individual particles of said active material may be separated from one another by a distance of at least 50 μm, suitably at least 70 μm, preferably at least 90 μm, more preferably at least 110 μm, especially at least 120 μm. Said particles may be separated from one another by a distance of less than 1000 μm, suitably less than 600 μm, preferably less than 400 μm, more preferably less than 200 μm, especially less than 150 μm. The aforementioned distance between particles suitably represents the pore singe of the internal region. Said active material is preferably held in position by a physical weld suitably provided by said inert material. Preferably, said inert material defines a random network in which said active material is embedded.

Said active material preferably includes accessible functionality so that covalent bonds can be formed between it and reagents used in the synthesis. Said active material may include at least 10 μmol, suitably at least 25 μmol, preferably at least 40 μmol, more preferably at least 55 μmol, especially at least 70 μmol of accessible functionality. In some situations, for example where said porous device is of the second type described, said active material itself is preferably porous and includes accessible functionality within its internal structure. Thus, preferably, the formation of covalent bonds between the active material and reagents used in the synthesis does not only take place at a surface of the active material, but also takes place within a solid portion of active material, for example within a bead of active material. Suitably, said active material includes a functional group able to participate in (preferably non-free radical) chemical reactions. For example, said active material may include a leaving group. Preferably, said active material is polymeric and is more preferably an organic polymeric material, for example a resin. Said active material is preferably a cross-linked resin. Said active material is preferably in the form of beads. Said active material is preferably non-cellulosic. Said resin may be a polystyrene-based resin (e.g. a substituted alkyl (for example methyl or ethyl) polystyrene, an aminomethylated polystyrene, a benzyloxybenzyl alcohol resin, a carboxypolystyrene, a polystyrene-divinylbenzene copolymer, a trityl chloride resin, a trityl resin, a phenoxy resin, a dihydropyran resin, a Merrifield resin, a formyl polystyrene, a benzhydrylamine resin, an oxime resin, a PEG polystyrene based resin) or a polyethylene glycol acrylamide (PEGA) resin. Said active material may be substituted methyl polystyrene, for example, chloromethyl polystyrene, hydroxymethyl polystyrene or aminomethyl polystyrene or a derivative of any of the aforesaid which incorporates a linker. Alternatively, said active material may be a substituted polypropylene (or other optionally-substituted polyalkylene polymer).

Such a polymer may be substituted with a haloalkyl, especially a chloromethyl, group or said active material may be a derivative of such a group which incorporates a linker.

Said active material may be a material which, in isolation, is swellable in organic solvents. Known active materials can swell from three to five times their original volume in solvents. However, advantageously, when incorporated in a porous device as described herein, the active material is restrained and may not significantly swell. As a result, the external shape of the porous device may be substantially unchanged, even during or after the device has been contacted with a solvent in which active material would normally swell. Furthermore, the size of the device may be substantially unchanged. In this regard, the maximum dimension of the device (e.g the length wherein the device is a cylinder) and/or any dimension may change by less than 80%, suitably by less than 60%, preferably by less than 40%, more preferably by less than 20%, especially by less than 10%, after the device has been immersed, for up to I hour, in a solvent in which the active material would normally swell.

Where the porous device includes an inert material, said inert material suitably does not participate in chemical reactions in said synthesis. For example, it preferably does not include a leaving group. Said inert material may be organic or inorganic. Said inert material is preferably non-cellulosic. Said inert material is preferably a sinterable material. Said inert material is suitably sinterable at a temperature of less than 500° C., preferably less than 400° C., more preferably less than 300° C., especially less than 200° C. Said inert material is preferably a sintered material. Said inert material is preferably a thermoplastic. Examples of organic material include organic polymeric materials which are suitably resins and may include, for example, optionally substituted, preferably unsubstituted, polyalkylenes (especially polyethylene and polypropylene), and polyhaloalkylenes (especially polyfluoroalkylenes such as polytetrafluoroethylene).

Said active material is preferably a comminuted material. Particles of said active material may be substantially spherical. Said active material may have particles of size in the range 10 μm to 100 μm.

Said porous device may include at least 10 wt %, suitably at least 20 wt %, preferably at least 10 wt %, more preferably at least 40 wt %, especially at least 45 wt % of active material. The amount of active material may be less that 90 wt %, suitably less than 80 wt %, preferably less than 70 wt %, more preferably less than 60 wt %, especially less than 55 wt %. For the avoidance of doubt, the aforementioned wt % of active material refers to the total amount of all active materials in said porous device. In some situations described hereinafter, a porous device may include more than one type of active material.

Said porous device may include at least 10 wt %, suitably at least 20 wt %, preferably at least 30 wt %, more preferably at least 40 wt %, especially at least 45 wt % of inert material. The amount of inert material may be less than 90 wt %, suitably less than 80 wt %, preferably less than 70 wt %, more preferably less than 60 wt %, especially less than 55 wt %. For the avoidance of doubt, the aforementioned wt % of inert material refers to the total amount of inert material in the porous device and includes a situation wherein more than one type of inert material is included.

Said porous device may include a filler or fillers. Said filler(s) may be coloured and different porous devices may include different colours thereby allowing different porous devices to be distinguished from one another.

Preferably, said porous device consists essentially of active material and inert material.

Said porous device suitably passes at least one, preferably any selection, more preferably all, of the following tests:
Test 1—The porous device is boiled in methanol. The device passes the test if it is unchanged after 10 minutes of boiling.
Tests 2 to 7—These are the same as Test 1 except the solvents are ethanol, 1,4-dioxan, water, DMSO, DCM, and THF respectively.
Test 8—The porous device is heated in DMF at 120° C. in an oil bath. The device passes the test if it is unchanged after 10 minutes treatment.
Test 9—The porous device is heated in DMF and centrifuged at 1300 r.p.m. The device passes the test if it is unchanged after 10 minutes of treatment.

Said porous device is suitably capable of supporting any one, preferably any selection, more preferably, all of the following reactions: a Suzuki reaction, a Mitsunobu reaction, alcohol oxidation using pyridine sulfurtrioxide in DMSO and reduction of an aldehyde to an alcohol using sodium cyanoborohydride.

According to a second aspect of the invention, there is provided a method of synthesising a plurality of different compounds, the method using a plurality of porous devices as described according to said first aspect and including contacting a first said porous device with a first sequence of reagents and contacting a second said porous device with a second sequence of reagents wherein said first and second sequences of reagents are different, thereby to prepare different compounds on said first and second porous devices.

Said first sequence of regents may comprise reacting said first porous device with, for example, an α-amino ester hydrochloride followed by a reduction and cyclisation reaction, followed by an alkylation reaction using an alkyl bromide. Said second sequence may be different from said first by using a different α-amino ester hydrochloride or a different alkyl bromide.

Preferably, the method according to second aspect is a method of synthesizing N different compounds, wherein N is a positive integer, using N porous devices, the method including using N different sequences of reagents and contacting said porous devices with a respective sequence thereby to prepare respective different compounds on said porous devices.

Integer N may be 4 or greater, suitably 10 or greater, preferably 20 or greater, more preferably 24 or greater. In some situations, N may be 50 or greater, suitably 100 or greater, preferably 200 or greater, more preferably 500 or greater, especially 1000 or greater.

Advantageously, the method may be used in any parallel array, “mix and split” or combinatorial technique. More particularly, the method may be used in techniques described in, for example, WO96/36436.

Preferably, the porous devices used in the method are, initially, substantially identical. Said devices, however, preferably include identifying means (or indicia) for uniquely identifying the devices from one another. The identification means may comprise, for example, numbers, letters, symbols or colours in a coded combination, Smiles strings, bar-codes, chemical structures, marked or printed punched card formats, ultravoilet-readable devices, or any other readable device, such as magnetic strips. In some embodiments, said identification means may comprise an electro-magnetically readable device, for example a device arranged to be read by an Rf transmitter or a magnetic readable device. Said identification means preferably includes an identifier, preferably an encoded identifier, arranged to be read by a form of reading device. The identifier preferably includes a unique code. The identity of the identifier and/or information associated with the identification means may be predetermined and/or not changeable after the identification means has been associated with the porous device.

According to a third aspect of the present invention, there is provided the use of a porous device as described according to said first aspect in the synthesis of a compound.

The invention extends to the use of a plurality of porous devices as described according to said second aspect in the synthesis of a plurality of compounds.

According to a fourth aspect of the present invention, there is provided a method of effecting an interaction between an active material and another material (hereinafter an “interacting material”), the method using a porous device comprising a body having an internal region which is porous, wherein said active material is entrapped within the internal region.

Preferably, the method comprises juxtaposing the active material and the interacting material, suitably in a fluid.

The porous device of the fourth aspect may include any feature of a porous device described in any statement herein.

The method may be for effecting a chemical interaction between the active material and said interacting material.

Said active material may be adapted to scavenge said interacting material from a fluid containing the interacting material. Suitably, the method includes the step of contacting the porous device with a fluid containing the material, suitably stirring the fluid to maximise contact between the interacting material and the device and, suitably, subsequently removing the porous device from the fluid after said active material of the device has scavenged the interacting material. Advantageously, the method may not involve filtration of the fluid containing the interacting material (i.e providing the porous device or porous devices in a fluid flow path of the fluid such that all of the fluid passes through a porous device) thereby obviating the need to handle the volume of fluid itself. However, in some embodiments, a said porous device or devices may act as active filters whereby said active interacting material interacts with material being filtered.

Said active material may be arranged to have an affinity for said interacting material. Said active material may have an affinity for particular metals, radioactive waste, resins, magnetic compounds or moieties, acids or bases.

Said active material may be a catalyst. Said active material may be chemical or biological.

Said active material may be adapted to interact with cells or enzymes, suitably thereby to immobilise the cells or enzymes for subsequent use.

Said active material may be a reagent which is arranged to interact with said interacting material and thereby cause a chemical reaction, preferably covalent bond formation, with said interacting material.

The method according to the fourth aspect may use a porous device which includes at least two different active materials entrapped within its internal region. The active materials may be as described in any statement herein. Advantageously, a said porous device may incorporate two active materials which would if contacted with one another in a fluid be reactive with one another; however, using such a porous device, the two active materials are spaced apart and thereby prevented from reacting.

Where a said porous device includes at least two different active materials, said at least two materials may comprise two materials selected from reagents, scavengers or catalysts. For example, a porous device may include a reagent and a scavenger; or two different scavengers or reagents etc.

The method may involve the porous device being placed in a column (or the like) through which fluid may flow. Advantageously, the column may include at least two different types of porous device (i.e containing different active materials) which may be arranged to define a mixed bed in the column or may be arranged sequentially. The devices may, suitably, be disc-shaped for use in a column. Advantageously, separate porous devices including active materials which would, if contacted with one another in a fluid, be reactive with one another may be used with no detriment even if the porous devices contact one another.

The column described may be used in filtration. Alternatively, it may be used in continuous flow synthesis, wherein, suitably, the column is sequentially packed with porous devices adapted to interact with material at stages of the synthesis.

In another embodiment, a plurality of porous devices may be arranged in an array. Each porous device may include a different active material and each active material may be arranged to interact with a different interacting material. The array may then be used for affinity purification. Preferably, the array is a combinatorial matrix array.

According to a fifth aspect of the present invention, there is provided a porous device comprising a body having an internal region which is porous, wherein an active material is entrapped, suitably substantially immovably, within the internal region.

Said porous device may be as described in any statement herein. It preferably comprises co-sintered active and inert materials.

Preferably, said active material is arranged to act as a support for a compound prepared in a solid phase synthesis. Preferably, said active material includes a linker. Preferably, said porous device includes a synthesized compound or a fragment thereof covalently bonded to the active material, for example said linker, and arranged to be cleaved from the device. Where said device is in the form of a sheet material, a plurality of different compounds may be covalently bonded to the device, for example, as a result of spot synthesis, suitably at spaced apart positions.

Said porous device preferably includes an identification means associated therewith, for example by being substantially permanently fixed to a part of the device.

According to a sixth aspect of the invention, there is provided a porous device comprising an active material which, in isolation, is swellable in an organic solvent, and a restriction means arranged to restrict swelling of the active material in said organic solvent.

Said solvent may be one of methanol, ethanol, 1,4-dioxan, water, DMF, DMSO, DCM, or THF.

Said restriction means may be of any type which is able to restrict swelling of the active material, suitably by at least 50%. However, said porous device may be of the second type described herein and said restrictor means may be provided by said inert material.

According to a seventh aspect of the present invention, there is provided a collocation or an assembly comprising a plurality of porous devices according to said fifth or sixth aspects.

Preferably, each porous device includes a unique identification means. Such a unique identification means is preferably provided even for porous devices which incorporate the same type of active material. Preferably, said identification means enables each porous device to be distinguished from every other porous device in the collocation or assembly.

The collocation or assembly may include at least 10, suitably at least 50, preferably at least 100, more preferably at least 1000, especially at least 5000 porous devices.

Said plurality of porous devices may be randomly arranged or arranged in an array which may, suitably, be one-dimensional or two-dimensional. Means for fixing each porous device in the array may be provided and this may simply comprise stringing members of the array together. Such an array may be arranged and/or manipulated, for example in the preparation of a library of compounds, as described in Applicant's co-pending PCT Application No. PCT/GB98/03875 or in WO96/16078 (Pfizer) and the contents of the aforementioned documents are incorporated herein by reference.

The invention extends to a collocation or assembly as described wherein the devices support a plurality of different compounds, suitably with one compound being supported per device. Preferably, a library of different compounds are supported by the devices.

According to an eighth aspect of the present invention, there is provided a method of synthesizing a library of compounds, the method using a plurality of porous devices according to said fifth or sixth aspects and suitably including the step of subjecting each porous device to a unique sequence of treatments and/or reactions, thereby to prepare different compounds on the porous devices.

The method may further include the step of cleaving the compounds synthesized from the devices.

According to a ninth aspect of the present invention, there is provided a method of manufacturing a porous device for use as described in any statement herein, the method comprising causing a body having a porous internal region to form with an active material entrapped therewithin.

Porous devices of said first type described above may be made by mixing a material which is to define the active material, suitably in powder form, with a removable pore-forming material; forming the mixture into a desired shape; causing agglomeration of the mixture, for example by sintering (or otherwise heating) the mixture, optionally under applied pressure; and, thereafter, removing the pore-forming material.

Said pore-forming material may be removed by causing its decomposition. Such a pore-forming material may be calcium carbonate. Alternatively, said pore-forming material may be removable by dissolution, for example by contacting the agglomerated mixture with a solvent. Such a pore-forming material may be sodium chloride which may be removed by dissolution in water. It will be appreciated that the amount of pore-forming material relative to active material may be adjusted, thereby to adjust the porosity of the porous device.

The material which is to define the active material of the first type of porous device is preferably thermoplastic and/or preferably sinterable.

In some situations, a porous device of the first type may be prepared by sintering comminuted active material in the absence of a pore-forming material.

It may be possible to prepare a porous device of said first type, wherein the material which is to define the active material is functionalized after preparation of the porous structure, thereby to define the active material. Such functionalisation may be effected by radiation grafting, for example as described in PCT/GB98/03875. However, it is preferred that the material which is to define the active material is an active material prior to preparation of said porous structure (and the material does not need to be post-functionalized to make it into an active material); for example, it may be a resin have available functionality, which functionality may by provided on a polymeric material by suitable means, for example, radiation grafting as described. An especially preferred material of this type may be polypropylene which has been radiation grafted to define functionality thereon.

Porous devices of the second type described above may, in one embodiment, be made by mixing inert material, suitably in powder form, with active material, suitably in powder form; forming the mixture into a desired shape; and causing agglomeration of the mixture, for example by sintering (or otherwise heating) the mixture, suitably in a mould, optionally under pressure. Said sintering/heating is preferably carrier out at a temperature below the melting point (and/or at a temperature below that at which flow begins) of the active material. Said sintering/heating is preferably carried out at a temperature not lower than the softening temperature of the inert material and not higher than the decomposition temperature thereof. Said sintering/heating preferably takes place at or about (for example within 10% of) the melting point of the inert material. Thus, preferably, in general terms, said porous devices of the second type are made by co-sintering a mixture of an inert and an active material.

In another embodiment, porous devices of the second type may be made by in situ polymerisation, in the presence of active material, of monomers to provide, when polymerised, the inert material, whereby foaming is effected during polymerisation and the process is such that the active material becomes distributed throughout the foamed inert material.

In a further embodiment, a porous material may be impregnated with an active material and, optionally, steps may be taken to immobilise the active material within the device.

The method of the ninth aspect may include causing an active material which includes a linker as described herein to be entrapped. Either the linker may be an integral part of the active material used in the preparation of the porous device or a porous device including an active material may be post-functionalized to provide said linker.

Where the method is for making a porous device according to the fourth aspect, it may be advantageous to post-treat a device manufactured as described herein thereby to provide a desired active material in the device.

A preferred method for manufacturing any porous device described herein which includes an active material and inert material involves co-sintering a mixture comprising particles of said active material and said inert material, thereby to provide a monolithic structure. The method may be of particular utility wherein the active material is of a type described according to the first aspect or is a catalyst (suitably for catalysing a solution phase reaction) or is a solid support reagent, suitably wherein the reagent is for use in a solution phase reaction.

Said inert material is preferably a thermoplastic.

In an exemplary embodiment, the present invention relates to a method of preparing new materials suitable for use as substrates in solid phase chemistry, and materials obtained thereby.

The method of preparing the new materials involves the process of co-sintering a chemically active species bearing or containing accessible functionality with a variety of matrix-forming materials. The matrix-forming materials may additionally, in themselves, bear chemically functionality.

The method of sintering may be as follows: An intimate mixture of an organic or inorganic matrix forming material and a number of chemically active species, bearing or containing accessible functionality, is first formed into an appropriate physical shape. The new mixture is then sintered or co-sintered by subjecting it to a variable temperature for a variable residence time according to the melt-flow characteristics of the matrix forming material. A unique identifier can, if necessary be incorporated during formation, or applied post manufacture.

The support materials which can be co-sintered include, without limitation: polystyrene based resin beads of the type included below, polypropylene based materials as resin beads and powders, chemically modified beads and powders, zeolites, Teflon beads or any inorganic and organic powder or bead which will allow chemical or physical attachment to their surface or to their interior of active chemical reagents or molecules.

A selection of support materials suitable for sintering include: Oxime resin, Wang resin, NovaSyn TB amino resin, p-Nitrophenyl carbonate Wang resin, Aminomethyl-NovaGel HL, 2-Chlorotritylchloride resin, 3,5-Dimethoxy-4-formyl-phenoxyethoxy-methyl polystyrene, Merrifield Resin LL, Zeolites, 5A, 4A, 3A, 2A molecular sieves, Montmorillite clay powder and Amberlyst.

Co-sintering matrix forming materials may include, without limitation any organic or inorganic matrix forming material of appropriate melt-flow characteristics such as to permit formation via physical attachment or containment of support materials detailed above. This includes is without limitation, polyethylene, polypropylene, per halo-polyalkylenes and other chemically and physically suitable materials.

Materials of the type described are suitable, in various forms for chemistry, as follows:
In solid phase chemistry, where the support can be the matrix trapped resin beads included therein or the matrix itself.
For use in solution phase chemistry where substrates in solution may be induced to react together by a reagent or number of reagents trapped in or chemically attached to a solid phase, itself trapped within the matrix. The matrix itself can be also have a reagent or number of reagents trapped or chemically attached to its surface.
For catalysing various chemical reactions by occluded reagents entrapped within, for example an inorganic zeolite matrix, itself entrapped within the sintered matrix, or the matrix itself could be the catalyst.

The exemplary embodiment includes the possibility of sintering an existing polymeric material in powder or bead form, which itself has been chemically or physically modified, to allow attachment or entrapment of active chemical species on or within the surface of the powder or bead form into new physically constrained shapes. These include without limitation cylinders, rods, sheets, capsules, tablets, plugs, streamers, tapes, etc.

The embodiment allows the incorporation of a unique identifier at the point of manufacture of new sintered physical forms or identification subsequent to manufacture. This includes without limitation indicia which uniquely characterise each reaction zone. The indicia may comprise, for example, numbers, letters, symbols or colours in a coded combination. The indicia may be applied to the respective reaction zones before synthesis commences using known printing methods. These are preferably such that the ink used will not leach out of the reaction zones during the synthetic procedures, or otherwise interfere with formation and subsequent removal of a compound held on a particular reaction zone. UV sensitive ink which is “fixed” to the reaction zones by exposure to ultraviolet radiation after printing is generally suitable for this purpose. Other types of indicia, not necessarily optical in nature, may be used for identifying individual reaction zones. Possible alternatives include Smiles strings, bar-codes, chemical structures, marked or printed punched card formats, ultraviolet-readable fluorescent systems and electro-magnetically readable devices such as magnetic strips and RF ID, snowflakes dot matrix reading and other analogous systems. The type of indicia used may depend on the size and shape of the support material and/or reaction zones.

An additional principle is that one or more layers of reagent containing matrices may be simultaneously or subsequently formed or reformed to provide a material containing 2 or more reagent matrices within the same physical format thereby allowing 2 or more reactions to proceed concurrently or sequentially within the same matrix.

Any feature of any aspect of any invention or embodiment described herein may be combined with any feature of any aspect of any other invention or embodiment described herein.

Specific embodiments of the invention will now be described, by way of example, with reference to the accompanying figures, wherein:

FIG. 1 is a schematic representation of a porous plug;

FIG. 2 is a cross-section along line II-II of FIG. 1;

FIG. 3 is an electronmicrograph of a section through the plug at a first magnification; and

FIG. 4 is an electronmicrograph of a section within box III of FIG. 2, at a higher magnification.

The porous plug 2 of FIGS. 1 and 2 comprises an inert carrier and a functionalized resin which have been sintered together under pressure in a mould to define a self-supporting rigid cylindrical structure which has substantially constant density and porosity across its extent. The plug can be used in many applications, for lo example in the synthesis of chemical compounds which can be covalently attached to the functionalized resin. Further details are provided below.

A typical process for manufacturing a plug 2 involves intimately mixing micronized ultra-high molecular weight polyethylene (to provide the inert carrier) and beads (e.g of particle diameters in the range of 10-100 μm) of a functionalized resin. The ratio of the weight % of polyethylene to functionalized resin is suitably about 1. The mixture of polyethylene and resin is poured into a mould made of aluminium alloy, with care being taken to ensure consistent packing of the mould. The mould is then placed in an oven and sintered at 190° C. for 20-25 minutes in ambient atmosphere. After removal of the mould from the oven, it is allowed to cool and the plug is then removed from the mould.

Whilst any size or shape of plug could be produced, the cylinder produced and used as described herein has a diameter of 6 mm, a height of 9 mm and a volume of 255 mm². Such plugs were specifically made for use, in 96 well plates which are conventionally used in organic synthesis.

In the electronmicrographs of FIGS. 3 and 4, the spheres are the functionalized resin and the material between the spheres is the inert carrier. It will be noted that the inert carrier defines a matrix which physically holds or supports the spheres of functionalized resin in spaced-apart fixed positions—there are no covalent bonds formed between the functionalized resin and the inert carrier. It will also be noted that the internal structure of the plug is highly porous and that substantially all of the surface area of the spheres of functionalized resin is freely exposed (i.e not covered with inert carrier) so that the majority of the functionalized resin is available for subsequent chemical reactions.

A range of plugs (A to J) have been made using the general procedure described above and incorporating one of the functionalized resins detailed in Table 1.

| | | Calbiochem- | | | |
|---|---|---|---|---|---|
| | | Novabiochem | Loading in | | |
| | | (UK) | mmole/g on | | |
| Plug | Functionalized | LtdCatalogue | the starting | | |
| Identifier | Resin | No. | resin | Particle size | |
| A | NovaSyn TG Resin | 01-64-0043 | 0.27 | 90 | μm |
| B | 3,5-Dimethoxy-4- | 01-64-0261 | 0.96 | 100-200 | mesh |
| | formyl- | | | | |
| | phenoxyethoxy- | | | | |
| | methyl polystyrene | | | | |
| C | 2-chlorotritylchoride | 01-64-0114 | 1.14 | 100-200 | mesh |
| | resin | | | | |
| D | NovaSyn TG amino | 01-64-0094 | 0.3 | 130 | μm |
| | resin | | | | |
| E | Merrifield Resin LL | 01-64-0008 | 0.96 | 100-200 | mesh |
| F | Rink Acid resin | 01-64-0012 | 0.52 | 100-200 | mesh |
| G | Wang resin | 01-64-0014 | 0.83 | 100-200 | mesh |
| H | Oximo resin | 01-64-0022 | 0.57 | 200-400 | mesh |
| I | p-Nitrophenyl | 01-64-0123 | 0.54 | 100-200 | mesh |
| | carbonate Wang | | | | |
| | resin | | | | |
| J | Aminomethyl- | 01-64-0283 | 0.76 | 90 | μm |
| | NovaGel HL | | | | |

It has been found, in general, that the plugs described can be used in any situation where the functionalized resin may be used since the plugs are both physically and chemically stable and the functionalized resin can be accessed by reagents. Examples of treatments/reactions undertaken using the plugs are described below.

### EXAMPLE 1

Samples of each of plugs A to J were boiled for 10 minutes in each of the following solvents: methanol, ethanol, 1,4-dioxan, water, DMF (test undertaken using an oil bath at 120° C.), DMSO, DCM and THF. The plugs were subsequently examined and found to be unchanged by the treatment.

### EXAMPLE 2

Samples of each of the plugs A to J were suspended in DMF and centrifuged for 10 minutes at 13000 r.p.m. The plugs were unchanged by the treatment.

### EXAMPLE 3

Ten different pre-loaded resins (PS and PS-PEG's) and a range of different linkers (eg formyl Wang and Trityl linkers to Oxime based supports) were prepared in plug form. Their synthetic utility was demonstrated in comparison to identical batches of loose functionalized resin under a range of loading, synthesis and cleavage procedures. In all cases, products isolated after cleavage from the functionalized resin of the plugs, in terms of purities and yields, were comparable to those of the loose resin.

### EXAMPLE 4

A broad range of typical literature solid phase chemistries were carried out on plugs A to J. These ranged from the formation of aminomethyl and Wang resin from chloromethyl resin (Plug E), to Suzuki chemistries, reductive aminations, reductions, oxidations, acylations, esterifications, 1,3-dipolar additions, SN-displacements and benzodiazepine synthesis. In all cases, little (if any) adaptation from traditional resin synthetic protocols were needed.

### EXAMPLE 5

### Derivatisation of Chloromethylstyrene Resin (Merryfield Resin LL) to Aminomethyl Resin

One plug of chloromethyl Merrifield resin (78 μmol) (Plug E) was suspended in dry DMF (10 ml). Potassium phthalimide (10 eq) was added and the reaction stirred gently at 120° C. for 24 hours. The plug was washed with hot DMF (5×5 ml), DMF:H₂O (1:1, 5×5 ml), dioxan: H₂O (1:1, 5×5 ml), MeOH (5×5 ml), DCM (5×5 ml), and ether (5×5 ml). The plug was then suspended in ethanol (10 ml), hydrazine hydrate (2 ml) was added and the reaction refluxed for 4 hours. It was then washed as above and analysed to give a substitution of 55.8 μmol (71% conversion based on manufacturer's loading).

### EXAMPLE 6

### Preparation of a Resin-Bound Acid-Labile Fmoc-Rink Linker

Encapsulated aminomethylated polystyrene resin (1 plug, 55.8 μmol) prepared in Example 5, previously swollen in DCM, was treated with a solution of p-{(R,S)-(-[1-(9H-fluoren-9-yl)-methoxyformamido]-2,4-dimethoxybenzyl}-phenoxyacetic acid (1.5 eq), DIC (1.5 eq) and HOBt (1.5 eq) in DCM (5 ml) and the reaction shaken for 48 hours. The plug was washed with DCM (5×5 ml), DMF (5×5 ml), MeOH (5×5 ml), and Et₂O (5×5ml). (referred to from here on as “the usual wash cycle”). The remaining free amino sites were capped with excess acetic anhydride/pyridine in DCM. The plug was again washed according to the usual wash cycle and a quantitative Fmoc test gave a substitution of 40 μmol (75% yield).

### EXAMPLE 7

### Derivatisation of the Chloromethyl Resin with a Wang Linker

One plug of the Merrifield chloromethyl resin (78 μmol) (Plug E) was suspended in acetonitrile (10 ml). 4-Hydroxybenzaldehyde (0.2 g, 1.6 mmol), K₂CO₃ (0.4 g, 1.6 mmol) were added followed by sodium iodide (0.24 g, 1.6 mmol). The mixture was refluxed for 48 hours. The plug was washed according to the usual wash cycle and then suspended in MeOH (5 ml). Sodium cyanoborohydride (10 eq) was added and the reaction stirred gently. A trace of bromocresol green was added to monitor the pH which was maintained by periodic addition of 10% HCl in ethanol (one or two drops at a time). After 16 hours, the plug was washed to produce the resin derivatised with Wang linker.

### EXAMPLE 8

### Preparation of Fmoc-Phe-Gly-OH

The Wang derivatised plug prepared in Example 7 was reacted with Fmoc-Gly-OH (0.3 g, 1 mmol), DIC (1 mmol), DMAP (0.1 mmol) in DCM (10 ml) for 24 hours. After washing, a quantitative Fmoc test gave a substitution of 20 μmol. The plug was then coupled to Fmoc-Phe-OH (0.4 mmol), DIC (0.4 mmol) and HOBt (0.4 mmol) in DCM for 24 hours. After washing, the plug was treated with 95% TFA and the crude product analysed by HPLC to reveal the desired dipeptide [ES-MS m/z=445 (M+H)) as the only major compound.

### EXAMPLE 9

### Preparation of the Tripeptide Fmoc-Ala-Phe-Gly-NH₂

One plug of the Fmoc-Rink linker resin of Example 6 was treated with 20% piperidine in DMF for 20 minutes. After the usual wash cycle, Fmoc-Gly-OH (Seq), HOBt (5 eq) and DIC (5 eq) were added and the coupling allowed to proceed for 4 hours in DCM (10 ml). Some precipitation was observed as the reaction proceeded and 1 ml DMF was added to get a clear solution. After the usual wash cycle and the removal of the Fmoc group, the analogous procedure was used to couple Fmoc-Phe-OH and then Fmoc-Ala-OH to obtain Fmoc-Ala-Phe-Gly-Rink linker resin. The tripeptide was then cleaved from the resin by shaking with 95% TFA for 1 hour. Volatiles were removed under vacuum and the crude tripetide purified by semi-preparative HPLC to provide 10.4 mg of pure product (63% yield). The product was identical to that obtained on ordinary polystyrene beads.

### EXAMPLE 10

### The Suzuki Experiment

Three plugs of the Fmoc-Rink linker resin of Example 6 (120 μmol) in total were treated with 20% piperidine in DMF for 20 minutes. After the usual wash cycle, Fmoc-Gly-OH (5 eq), HOBt (5 eq) and DIC (5 eq) in DCM/DMF (20/2 ml) was added. The reaction was shaken for 6 hours. A Ninhydrin test was negative. The Fmoc group was removed and the resulting amino product was coupled to 4-iodobezoic acid (5 eq), DIC (5 eq) and HOBt (5 eq) in DCM (20 ml). The reaction was shaken for 18 hours. After washing, the plugs were swollen in DMF (20 ml) for 10 minutes. To these were then added phenylboronic acid (1.5 eq), Pd[P(Ph)₃]₄ (0.1 eq) and K₂CO₃ (2 eq). The mixture was gently stirred and heated at 100° C. for 24 hours. The plugs became black, but they washed well using the usual wash cycle. The plugs were then treated with 95% TFA. HPLC analysis of the crude revealed the desired product (80% pure) which was purified and isolated (51% yield)

### EXAMPLE 11

### The Mitsunobu Experiment

Two plugs of the Fmoc-Rink linker resin of Example 6 (80 μmol in total) were treated with 20% piperidine in DMF for 20 minutes. After the usual wash cycle, Ac-Tyr-OH (5 eq), HOBt (5 eq) and DIC (5 eq) were added and the coupling allowed to proceed for 6 hours in DCM/DMF (20/2 ml). The plugs were washed according to the usual wash cycle followed by dry THF (5×) and then suspended in dry THF (10 ml). Triphenylphosphine (5 eq) was added followed by dry benzyl alcohol (10eq). Then, diethyl azodicarboxylate (5 eq) dissolved in dry THF (5 ml) was added in five portions at 5 minute intervals. After 2 hours, the plugs were washed according to the usual wash cycle. Cleavage with 95% TFA and analysis of the crude by HPLC revealed two components, which were separated on semi-prep HPLC. The major component (42% yield) had the required molecular mass as shown by ES MS. It also co-eluted with the same product prepared on ordinary polystyrene beads in the same manner.

### EXAMPLE 11

### Oxidation Experiment

Two plugs of the Fmoc-Rink linker resin of Example 6 (80 μmol in total) were treated with 20% piperidine in DMF for, 20 minutes. After the usual wash cycle, Fmoc-Phe-OH (5 eq), HOBt (5 eq) and DIC (5 eq) were added and the coupling allowed to proceed for 6 hours in DCM (20 ml). After removal of the Fmoc group, the same procedure was used to couple 4-(hydroxymethyl)benzoic acid. One plug was treated with 95% TFA to provide the starting material. The second plug was suspended in dry DMSO (10 ml) and to this was added pyridine sulfur trioxide (10 eq), triethylamine (10 eq), and the reaction was shaken for 18 hours. After the usual wash cycle, and cleavage with 95% TFA, the crude was analysed on HPLC to reveal complete conversion to the aldehyde product (confirmed by ES MS and co-elution with an authentic sample, 53% yield).

### EXAMPLE 12

### Reduction Experiment

Two plugs of the Fmoc-Rink linker resin of Example 6 (80 μmol in total) were treated with 20% piperidine in DMF 20 minutes. After the usual wash cycle, Fmoc-Phe-OH (5 eq), HOBt (5 eq), and DIC (5 eq) were added and the coupling allowed to proceed for 6 hours in DCM (20 ml). After removal of the Fmoc group, the same procedure was used to couple 4-carboxybenzaldehyde. One plug was treated with 95% TFA to provide the starting material. The second plug was suspended in MeOH (10 ml) and treated with sodium cyanoborohydride (10 eq). A trace of bromocresol green was added to follow the reaction. An acidic medium was maintained (yellow colour) by periodic addition of 10% HCl in ethanol (one or two drops). After 4 hours, the plug was washed according to the usual wash cycle and then cleaved with 95% TFA. HPLC analysis of the crude revealed incomplete conversion. The trace showed a peak corresponding to starting aldehyde, the cyanohydrin, and the desired alcohol product. These were all separated on semi-prep HPLC. Yield of alcohol 44%.

### EXAMPLE 13

### Ease of Washing of Encapsulated Polystyrene Resin

One plug of the type prepared in Example 5 (55.8 μmol) and an equivalent amount of aminomethyl TentaGel resin beads were separately treated with two equivalents of bromophenol blue in DCM(10 ml). Both materials became the same intensity blue colour by eye. The two materials were washed in parallel with equal volumes of 10% triethylamine in DCM. TentaGel resin required 5 washes (10 ml each) to become colourless while the encapsulated resin of Example 5 required 12 washes. Similarly, both resins were treated with two equivalents of methyl red and washed in the same way. TentaGel resin required 3 washes to become colourless while the encapsulated resin of Example 5 required 5 washes.

### EXAMPLE 14

### Preparation of Fmoc-Phe-NH—OH

One plug of Rink acid derivatised resin (40 μmol) was suspended in dry DCM. The reaction was gently stirred and a freshly prepared solution of 1% HCl in DCM/THF (3:1) (9 ml) was slowly added. The plug was then washed with DCM (2×), THF (2×), and DCM (2×). The plug was immediately suspended in DCM (10 ml) and then Fmoc-NH-OH (1.5 eq) and DIEA (1.5 eq) were added and the reaction left stirring for 24 hours. After washing the plug according to the usual wash cycle, the Fmoc group was removed and the resulting amino compound coupled to Fmoc-Phe-OH (5 eq), DIC (5 eq), and HOBt (5 eq). The coupling was allowed 24 hours. The plug was washed and then treated with 5% TFA/DCM to provide the carboxamic acid Fmoc-Phe-NH—OH (51% yield, 68% pure).

### EXAMPLE 15

### Preparation of Ac—NH—CH₂Ph

One plug of 3,5-dimethoxy-4-formyl-phenoxyethoxy-methyl polystyrene resin (81 μmol) (i.e. Plug B) was suspended in dry trimethyl orthoformate (6 ml). Benzylamine (10 eq) was added and the reaction stirred gently at 70° C. for 3 hours. The plug was then washed with dry DMF (3×) and dry MeOH (3×). It was then suspended in dry MeOH (5 ml) and reacted with NaBH₄ (5 eq) for 24 hours. The plug was carefully washed with MeOH (5×) and DCM (5×). It was then acetylated with acetic anhydride/pyridine in DCM for 3 hours. After washing, it was cleaved with 25% TFA/DCM for 1 hour to provide the title compound (50% yield, 80% pure).

### EXAMPLE 16

### Preparation of p-CH₃—C₆ H₄—CH₂—CO—NH— (CH₂)₃—NH₂

One plug of 2-chlorotritylchoride resin (97 μmol) (i.e Plug C) was reacted with 1,3-diaminopropane (10 eq) in DCM for 24 hours. It was washed and then coupled to p-tolyacetic acid (5 eq), DIC (5 eq), HOBt (5 eq) in DCM for 6 hours. After washing, the plug was treated with 50% TFA in DCM to provide the title compound (49%, 70% pure).

### EXAMPLE 17

### Library of 20 3,4-Disubstituted-7-carbamoyl-1,2,3,4-tetrahydroquinoxalin-2-ones

(i) Attachment of 4-Fluoro-3-Nitrobenzoic Acid to Rink Amide Resin

Twenty plugs of the Fmoc-Rink linker-resin (40 μmol each) prepared in Example 6 were treated with 20% piperidine in DMF for 20 minutes. The plugs were washed with DMF (3×), MeOH (3×), DCM (3×), and Et₂O (3×) and dried in vacuo. To the dried plugs was then added DIPEA (5 eq in 10 ml DCM) followed by 4-fluoro-3-nitrobenzoyl chloride (5 eq, in 10 ml DCM; freshly prepared by reaction of the acid with oxalyl chloride). The reaction was gently stirred for, 6 hours after which a ninhydrin test was negative.

(ii) General Procedure for Aromatic Substitution of the Aryl Fluoride with α-Amino Esters

The twenty plugs were split into four groups (five plugs each). To each group was then added 10 equiv of α-amino ester hydrochloride (L-alanine methyl ester, L-leucine methyl ester, L-phenylalanine methyl ester, and L-phenylglycine methyl ester), 20 equiv of DIPEA and 10 ml DMF at room temperature. The suspensions were shaken for 3 days. The supernatants were removed and the plugs were washed as above and dried.

(iii) General Procedure for Reduction of the Aryl Nitro Group and Cyclisation

To each group of five plugs was added 20 equiv of SnCl₂.2H₂O and 10 ml of DMF. The suspensions were shaken for 3 days. The supernatants were removed and the resins washed as above and dried.

(iv) General Procedure for Alkylation at N-4 Position of the Quinoxalinone with Alkyl Halides

To each plug of each group of the cyclised resins were added 25 equiv of alkyl bromide [benzyl bromide, 4-nitrobenzyl bromide, 2-(bromomethyl)naphthalene, 4-methylbenzyl bromide and methyl 4-(bromomethyl)benzoate], 25 equiv of K₂CO₃ and 2 ml of acetone. The 20 reactions were then heated at 55° C. for 48 hours. The plugs were then washed with acetone (3×), H₂O (3×) DCM (3×), and Et₂O (3×) and then dried in vacuo.

(v) General Cleavage Procedure

To each plug was added 5 ml of 95% TFA and the mixture allowed to stir for 1 hour. Supernatants were separated and the plugs were washed with MeOH (3×2 ml). The combined supernatants were concentrated and the crude products analysed by HPLC and MS.

### EXAMPLE 18

### Library of 25 Biaryl Derivatives

(i) Twenty-five plugs of the Fmoc-Rink linker-resin (40 μmol each) prepared in Example 6 were treated with 20% piperidine in DMF for 20 minutes. The plugs were washed with DMF (3×), MeOH (3×), DCM (3×), and Et₂O (3×) and dried in vacuo. The plugs were divided into five groups (five plugs each). To each group in DCM/DMF (20/2 ml) was added 5 equiv of one of five iodo-aryl carboxylic acids (4-iodobenzioc acid, 3-iodo-4-methylbenzoic acid, 2-iodohippuric acid, 4-iodophenylacetic acid and 4-iodophenoxyacetic acid), DIC (5 eq), and HOBt (5 eq). The reactions were shaken for 24 hours. The plugs were thoroughly washed with DMF (5×), DCM (5×), MeOH (5×), and Et₂O (5×). Each plug of each group was separately swollen in 3 ml dry DMF. To each plug was then added 1.5 equiv of one of five boronic acids (phenylboronic acid, 4-methylphenylboronic acid, 3-acetylphenylboronic acid, 4-methoxyphenylboronic acid, and thiopene-2-boronic acid), Pd[P(Ph)₃]₄ (0.1 eq), and K₂CO₃ (2 eq). The reactions were heated at 100° C. for 24 hours. The plugs were washed with hot DMF (5×), MeOH (5×), DCM (5×), and Et₂O (5×).

(ii) Cleavage and Analysis

To each plug was added 5 ml of 95% TFA and the mixture allowed to stir for 1 hour. Supernatants were separated and the resins were washed with MeOH (3×2 ml). The combined supernatants were concentrated and the crude products analysed by HPLC and MS.

### EXAMPLE 19

### Library of 24 Substituted Pyrrolidines

(i) Attachment of the Wang Linker

Twenty-four plugs of the aminomethyl polystyrene resin (55.8 μmol/plug) prepared in Example 5 were swollen in DCM/DMF (50/10 ml). To these were added 5equiv of 2-[4-(hydroxymethyl)phenoxy]acetic acid, DIC (5 eq), and HOBt (5 eq). The mixture was shaken for 24 hours. The plugs were then washed with DMF (5×), MeOH (5×), DCM (5×) and Et₂O (5×)

(ii) Attachment of the Amino Acids

The plugs were divided into four groups (6 plugs each). Each group was then reacted with 5 equiv of one of four Fmoc-amino acids (Fmoc-Gly-OH, Fmoc-Ala-OH, Fmoc-Leu-OH, and Fmoc-Phe-OH), DIC (5 eq), and DMAP (0.1 eq). The reactions were allowed 24 hours. The plugs were then thoroughly washed as above.

(iii) Formation of the Resin-Bound Aryl Imines

The plugs of each group were separately treated with 20% piperidine in DMF for 20 minutes. They were washed as above and dried in vacuo. Each group of plugs was in turn divided into three lots (2 plugs each); thus 12 separate reactions. Each lot was suspended in dry 1% AcOH/DMF (4 ml) and to this was added 10 eq of one of three aldehydes (benzaldehyde, o-tolyldehyde, and 2-methoxybenzaldehyde). Unreacted amines on the plugs were capped with an excess of Ac₂O, DIPEA in DCM for one hour. The plugs were thoroughly washed and dried.

(iv) 1,3 Dipolar Cycloaddition of Resin-Bound Azomethine Ylides

One plug from each lot was placed in a separate vial; thus 24 separate reactions. To each plug was then added 2 ml of a 1M AgNO₃ solution in MeCN, and 1 ml of a 1M NEt₃ solution in MeCN followed by 1 ml of a 1M solution in MeCN of one of two olefins (acrylonitrile and methyl acrylate). The 24 vials were stoppered and shaken for 24 hours. The plugs were then washed with MeCN (5×), DMF (3×), DCM (3×), MeOH (3×), and Et₂O (3×).

(v) Cleavage and Analysis

To each plug was added 5 ml of 95% TFA and the mixture allowed to stir for 1 hour. Supernatants were separated and the resins were washed with MeOH (3×2 ml). The combined supernatants were concentrated and the crude products analysed by HPLC and MS.

The above reactions illustrate how the functionalized s resins of the plugs are available for involvement in the synthesis of compounds, whereby chemical compounds being synthesised are covalently linked to a reactive group on the resin. At the completion of the synthesis the compound synthesized can be cleaved from the resin.

As an alternative to the use of functionalized resins which act as solid supports for use in the preparation of compounds in the manner described, porous devices, for example plugs 2, comprising a carrier encapsulating an active material may be prepared and/or used as follows:
(a) The active material may be arranged to abstract a particular material from solution. Thus, the porous device is tailored to remove a predetermined material from a solution.
(b) The active material may be covalently linked to a material which has an affinity for a material that it is desired to remove from a fluid. The porous device may then be contacted with, for example, by being placed within, the fluid, whereupon the desired material is attracted to the material covalently linked to the active material by absorption/chemisorption. Suitably, the removal process simply involves an affinity between two materials and no actual exchange of material between the porous device and the fluid. Once sufficient of the material to be removed has been removed, the porous device can be removed from the fluid. Advantageously, the process described may obviate any need to filter the fluid, in a case where the material removed is a desired material and the remaining material is waste.
(c) The procedure of (b) may be adapted to remove metals or radioactive waste from fluids by suitable choices of active material and/or functionalization thereof.
(d) A ligand may be covalently bonded to the active material of a porous device and this arrangement may be used to entrap cells or enzymes.
(e) The active material may be a catalytic material or the active material may be functionalized by being bonded to another compound or moiety thereby to define catalytic material.
(f) The active material may be a reagent for use in a reaction, for example a resin-based reagent, or the active material may be derivatized to provide such a reagent.
(g) A single porous device may incorporate one scavenger for one material and one scavenger for another material. For example, one scavenger may be arranged to remove acid and one arranged to remove an amine. Such a device may be used to remove excess amine and acid from the reaction product of an acid and amine.
(h) In a variation on (g), the two scavengers may be provided by different porous devices. Other porous devices arranged to remove any other impurities may be used so that, after use of the devices, substantially uncontaminated amide product remains.
(i) A range of porous devices may be arranged to define a mixed bed in a column, thereby allowing a range of different materials to be simultaneously removed from a fluid stream using the column. Advantageously, the use of porous devices described allows the use of a range of active materials (or derivatized active materials) which would otherwise react together (or otherwise be incompatible and would therefore need to be kept apart).
(j) An array of porous devices each incorporating ligands of different affinities may be formed and used to determine the appropriate ligand to be used in an affinity column to remove a desired component from a mixture.
(k) In view of the fact that porous devices may include different reagents/materials which would usually be reactive to one another but when in the porous devices they will not react with one another, even if the two different porous devices are adjacent, a series of porous devices incorporating appropriate reagents may be arranged in a column and continuous flow synthesis effected by passing appropriate materials through the column.
(l) The active material or a derivative thereof may include a ligand arranged to allow a substance (eg a drug) to be detached therefrom over time. The porous device may then be arranged under the skin to allow slow leaching of the substance to the surrounding areas.

1-37. (canceled)

38. A method of synthesis of a compound attached to an active material, the method using a porous device comprising a body having an internal region which is porous, wherein an inert material which is a thermoplastic defines a porous support means and a multiplicity of particles of an active material are entrapped within the internal region in pores of said porous support means by co-sintering a mixture comprising particles of said inert material and said active material, wherein said pores of said porous support means are not defined by a fabric material, a filamentous material or a fibrous material and wherein said active material is a resin, the method of synthesis comprising the step of contacting said porous device with a first reagent under conditions which cause said first reagent to react with said active material, so that a bond is formed between the active material and said first reagent or a fragment thereof.

39. A method according to claim 38, wherein a said inert material which is a thermoplastic organic polymeric material defines said porous support means and a multiplicity of particles of said active material are entrapped within the internal region in pores of said porous support means by co-sintering a mixture comprising particles of said thermoplastic organic polymeric material and said active material.

40. A method according to claim 38, wherein said inert material comprises an unsubstituted polyalkylene.

41. A method according to claim 38, wherein said inert material is selected from polyetheylene or polypropylene.

42. A method according to claim 38, wherein said active material includes a functional group selected from a chloromethyl, hydroxymethyl or aminomethyl group.

43. A method according to claim 38, wherein said active material includes a linker or is covalently bonded to a linker in said synthesis, wherein said linker comprises a moiety to which a compound being prepared in said synthesis is bonded during its synthesis and wherein, after preparation of said compound, it may be cleaved from the active material by breaking a bond between the linker and said compound.

44. A method according to claim 38 which includes the step of cleaving from said active material a said compound attached to said active material.

45. A method according to claim 38, wherein said porous device is in form of a cylinder.

46. A method according to claim 45, wherein the length of the cylinder changes by less than 20% in a solvent in which the active material would normally swell.

47. A method according to claim 38, wherein said porous device has a smallest dimension of at least 5 mm.

48. A method according to claim 38, wherein said porous device has a dimension in a first direction of at least 5 mm; a dimension in a second direction perpendicular to said first direction of at least 5 mm; and a dimension in a third direction perpendicular to said first and second directions of at least 5 mm.

49. A method according to claim 38, where said porous device has a volume of at least 200 mm³.

50. A method according to claim 38, wherein said active material in said porous device includes at least 40 μmol of accessible functionality.

51. A method according to claim 38, wherein the maximum dimension of the porous device changes by less than 20% in a solvent in which the active material would normally swell.

52. A method according to claim 38, wherein said inert material does not participate in chemical reactions in said synthesis and does not include a leaving group.

53. A method according to claim 38, wherein said porous device includes at least 40 wt % of active material.

54. A method according to claim 38, wherein said porous device includes at least 30 wt % of inert material.

55. A method according to claim 54, wherein said porous device includes at least 40 wt % of active material.

56. A method according to claim 38, wherein said porous device consists essentially of active material and inert material.

57. A method according to claim 38, wherein said active material is selected from a substituted alkyl polystyrene, an aminomethylated polystyrene, a benzyloxybenzyl alcohol resin, a carboxypolystyrene, a polystyrene-divinylbenzene copolymer, a trityl chloride resin, a trityl resin, a phenoxy resin, a dihydropyran resin, a Merrifield resin, a formyl polystyrene resin, a benzhydrylamine resin, an oxime resin, a PEG polystyrene based resin or a polyethylene glycol acrylamide (PEGA) resin.

58. A method according to claim 38, wherein said active material is selected from chloromethyl polystyrene, hydroxymethyl polystyrene or aminomethyl polystyrene.
